(19) United States
(12) Patent Application Publication
(10) Pub. No.: US 2017/0363616 A1
(43) Pub. Date: Dec. 21, 2017

(54) PATTERNED NEUROMUSCULAR JUNCTIONS AND METHODS OF USE

(71) Applicant: Los Alamos National Security, LLC, Los Alamos, NM (US)

(72) Inventors: Rashi Iyer, Los Alamos, NM (US); Piyush Bajaj, Norwich, CT (US)

(73) Assignee: Los Alamos National Security, LLC, Los Alamos, NM (US)

(21) Appl. No.: 15/529,704

(22) PCT Filed: Nov. 24, 2015
(86) PCT No.: PCT/US15/62506

§ 371 Date: May 25, 2017

Related U.S. Application Data
(60) Provisional application No. 62/084,316, filed on Nov. 25, 2014.

(51) Int. Cl. G01N 33/50; C12M 3/00; C12M 1/12; A61N 1/05

(52) U.S. Cl. CPC G01N 33/5061; G01N 33/5058; A61N 1/05; C12M 21/08; C12M 25/08

(57) ABSTRACT

Disclosed herein are devices including a substrate, one or more regions of electrically active material, and optionally at least one electrode. Also disclosed herein are devices or systems including a substrate, one or more regions of electrically active material including muscle cells and neuronal cells, and optionally at least one electrode. In some embodiments, the muscle cells and neuronal cells form one or more neuromuscular junctions at defined locations on the electrically active material. Also disclosed are methods of using the devices and systems for analyzing the effect of compounds on neuronal cell, muscle cell and/or neuromuscular junction activity.

### CROSS REFERENCE TO RELATED APPLICATION

This claims the benefit of U.S. Provisional Application No. 62/084,316, filed Nov. 25, 2014, which is incorporated herein by reference in its entirety.

### ACKNOWLEDGMENT OF GOVERNMENT SUPPORT

This invention was made with government support under Contract No. DE-AC52-06NA25396 awarded by the U.S. Department of Energy. The government has certain rights in the invention.

### FIELD

This disclosure relates to devices and systems including patterned neuromuscular junctions and methods of use, such as for high throughput screening.

### BACKGROUND

The neuromuscular junction (NMJ) is a potent site of attack for a number of chemical and biological warfare toxins, for example animal toxins (such as snake venom), botulinum toxin, and organophosphorus compounds (such as sarin, soman, tabun, and O-ethyl S-[2-(diisopropylamino)ethyl] methylphosphonothioate (VX)). These compounds have been used as chemical warfare nerve agents in the last century. In addition, over 100 different organophosphorus compounds are currently used as insecticides and pesticides. These may contribute to injury to humans or animals exposed to the compounds. The NMJ is also affected in disease states, including myasthenia gravis and Lambert Eaton myasthenic syndrome.

### SUMMARY

Methods are needed for screening (particularly high throughput screening methods) for compounds that affect the NMJ, for example as countertoxins for naturally occurring toxins or organophosphorus or other chemical/biological warfare agents, as well as therapeutics for diseases impacting the NMJ. High throughput screening (HTS) methods require the ability to generate large numbers of NMJs in vitro. However, previous methods have relied on spontaneous formation of NMJs when motor neurons and myotubes are co-cultured. In addition to being a relatively rare event, the location of NMJ formation is not controlled. Disclosed herein are patterned NMJs (for example an array of NMJs on a graphene surface). These arrays have an advantage of providing a large number of NMJs that are present at defined locations, making HTS for compounds affecting the NMJ possible.

Disclosed herein are devices (in some examples referred to herein as “NMJ arrays”) including a substrate upon which is disposed at least one region of electrically active material, wherein the electrically active material is patterned to accommodate formation of one or more NMJs at a defined location. The device also includes at least one electrode connected to the region of electrically active material. In some embodiments, the region(s) of electrically active material have two or more areas with differing shape and/or orientation, such as a first area and a second area. In some embodiments, the region of electrically active material is patterned to include a first central area and at least a second area that is perpendicular to the first central area. In particular examples, the region of electrically active material includes a rectangular central first area and two or more (for example, 4, 6, 8, 10, 12, 16, 20 or more, such as 2-100, 4-50, 8-36, 2-40, 6-20, 10-80, 20-100) second areas that are oriented perpendicular to the first area and are in direct contact with the first area. In other examples, the second area (or third, fourth, fifth, sixth area, and so on, if present) is oriented radially to the first area, without necessarily being perpendicular to the first area.

Also disclosed herein are systems or assemblies including a substrate with at least one region of an electrically active material disposed on the substrate, a plurality of muscle cells (such as myoblasts and/or myotubes) and a plurality of neuronal cells (such as neuronal cells comprising neurites) on the at least one region of electrically active material, and at least one electrode connected to the region of electrically active material. In particular examples, the system or assembly also includes at least one NMJ between a muscle cell and a neuronal cell. In some embodiments, the region(s) of electrically active material have two or more areas with differing shape and/or orientation, such as a first area and a second area. In some examples, the muscle cells are present on one of the areas (such as the first area) and the neuronal cells are present on another area (such as the second area). The NMJ(s) may form between the muscle and neuronal cells at or near the point where the first and second areas meet or are in contact, providing a NMJ at a defined location.

Also disclosed herein are methods of using the devices or systems described herein, including methods of identifying a compound that affects activity of muscle cells, neuronal cells, and/or the NMJ. In some embodiments, the methods include contacting a disclosed device or system with one or more test compounds, stimulating the neuronal cells, and determining a response of one or more of the muscle cells, neuronal cells, and/or NMJs.

The foregoing and other features of the disclosure will become more apparent from the following detailed description, which proceeds with reference to the accompanying figures.

### BRIEF DESCRIPTION OF THE DRAWINGS

FIG. 1 is a schematic drawing showing an exemplary device (NMJ array) disclosed herein. The exemplary array (or “chip”) includes “islands” of electrically active material, such as graphene (gray patterns), patterned on a substrate (e.g., Si/SiO₂ surface). In exemplary systems disclosed herein, myotubes are formed on the central rectangular portion of the islands, and neuronal cells are grown on the perpendicular graphene extensions of the islands. The neuronal processes contact the myotubes and form NMJs. Each “island” of this exemplary embodiment is an individual sensor (e.g., a field effect transistor; FET), including gold electrodes (hatched boxes). In some examples, the graphene islands are patterned to include 6 or 10 NMJs per sensor. However, the islands can be patterned to include fewer or more NMJs, as desired. In the embodiment illustrated in this schematic, the array is about 2×4 cm and the FETs are about 160-170 μm apart. All units are μm, unless otherwise stated.

FIG. 2A is an exemplary embodiment showing a NMJ array with regions of electrically active material (sensors or FETs) patterned to include 6 NMJs each.

FIG. 2B is a series of illustrations of exemplary electrically active material patterned to include 4-8 NMJs each.

FIG. 2C is a schematic diagram of an exemplary electrical circuit for stimulating cells (such as myotubes) on a graphene surface and recording their electrical activity.

FIG. 2D is an exemplary multi-electrode array. Cells are cultured on electrically active material in the central “well” portion. Each electrode is individually addressable to a NMJ on the electrically active material.

FIG. 2E is an exemplary embodiment showing a NMJ array including microfluidic channels and means to introduce and remove fluids.

FIG. 3 illustrates exemplary methods for transferring graphene to a substrate (Si/SiO₂) and patterning the graphene with photolithography (adapted from Bajaj et al., Adv. Healthcare Mat. 3:995-1000, 2014).

FIGS. 4A-4C are a series of panels showing formation of myotubes on a graphene film in the presence or absence of insulin-like growth factor-1 (IGF-1). FIG. 4A is a series of digital images of C2C12 cells cultured in differentiation media (top) or differentiation media plus 50 ng/ml IGF-1 (bottom) at days 1, 3, and 5 of culture. Myotubes were visualized with a fluorescently labeled anti-myosin heavy chain (MHC) antibody and nuclei were visualized with 4′, 6-diamidino-2-phenylindole (DAPI). FIG. 4B is a graph showing the fusion index of C2C12 cells cultured in differentiation media or differentiation media plus 50 ng/ml IGF-1 at days 1, 3, and 5. FIG. 4C is a graph showing the cell density of C2C12 cells cultured in differentiation media or differentiation media plus 50 ng/ml IGF-1 at days 1, 3, and 5. *, p<0.05; **, p<0.01. Adapted from Bajaj et al., Adv. Healthcare Mat. 3:995-1000, 2014.

FIGS. 5A-5E are a series of panels showing formation of myotubes on graphene or SiO₂ surfaces in the presence or absence of IGF-1. FIGS. 5A and 5B are a series of digital images showing formation of myotubes in cultures of C2C12 myoblasts in differentiation media (FIG. 5A) or differentiation media with 50 ng/ml IGF-1 (FIG. 5B) on graphene (center) or SiO₂ (right) surfaces at days 2 and 4 of culture. The left most panels show the border (dashed line) between graphene (G) and SiO₂ (S) surfaces. Myotubes were visualized with a fluorescently labeled anti-MHC antibody and nuclei were visualized with DAPI. Fusion index (FIG. 5C), myotube area fraction (FIG. 5D), and cell density (FIG. 5E) were calculated for C2C12 myoblasts grown in differentiation media or differentiation media with 50 ng/ml IGF-lon graphene or SiO₂ surfaces at days 2 and 4 of culture. Adapted from Bajaj et al., Adv. Healthcare Mat. 3:995-1000, 2014.

FIG. 6 is a series of panels showing digital images of NSC-34 motor neuron cells grown in the indicated media for six days. Cells were visualized with anti-beta III tubulin (TUB3) antibody and DAPI (nuclear staining).

FIGS. 7A-7C are a series of graphs showing average number of neurites (FIG. 7A), mean neurite length (FIG. 7B), and sum of neurite length (FIG. 7C) in cultures of NSC-34 cells after six days in the indicated media.

FIGS. 8A-8C are a series of graphs showing neurite length per neuron (FIG. 8A), sum of neurite length (FIG. 8B), and number of neurites per neuron (FIG. 8C) in cultures of NSC-34 cells after the indicated number of days in Neurobasal media.

FIG. 9 is a schematic showing an exemplary protocol for co-culture and differentiation of myotubes (C2C12 cells) and neurons (NSC-34 cells) to produce NMJs.

FIGS. 10A-10C are a series of panels showing development of NMJs in co-cultures of C2C12 and NSC-34 cells using the protocol shown in FIG. 9. Cells were stained with anti-MHC, TUB3, and AchR antibodies, and DAPI. Co-culture of C2C12 and NSC-34 cells (FIG. 10A) showed development of NMJs as shown by presence of clusters of AchR (e.g., boxed areas), while no AchR clustering was seen in cultures of C2C12 cells only (FIG. 10B). FIG. 10C is a graph showing AchR staining intensity across ROI1 and ROI2 (boxes in FIG. 10A). Scale bar=100 μm.

FIG. 11 is a digital image showing formation of NMJs (AchR clustering) at the site of interaction of myotubes and neurons in co-culture. Scale bar=100 μm.

FIGS. 12A and 12B are digital images showing culture of NSC-34 cells and neurite formation on graphene (FIG. 12A) and graphene oxide (FIG. 12B). In FIG. 12B, the left two panels show fluorescent staining and the right panel is visualized with phase contrast. Scale bar =100 μm.

### DETAILED DESCRIPTION

HTS methods utilizing NMJs have been challenging because of the need for a large number of NMJs that can be analyzed simultaneously. Current methods for generating NMJs in vitro generally culture motor neurons and muscle cells together, leading to spontaneous NMJ formation which cannot be spatially controlled. In addition, the number of NMJs formed is low, typically around 1-4 NMJs in a 35 mm culture dish, which is a density of about 0.1-0.4/cm² (see, e.g., Umbach et al., PLoS One 7:e36049, 2012). Finally, analysis of the NMJ activity requires patch clamping of individual cells, which is time-consuming, analyzes a single cell at a time, and requires highly skilled technicians.

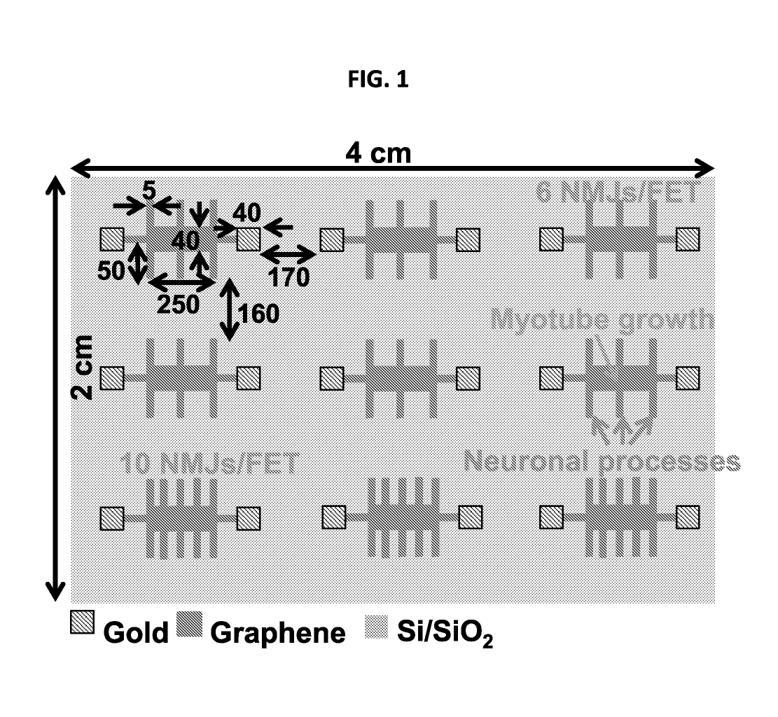

The inventors have developed devices with high density, spatially-controlled NMJs. The devices include one or more sensors created by patterning graphene or other electrically active materials on a substrate. In particular examples, graphene is used because it is electrically active and optically transparent (which is advantageous for use with cells that are electrically activated by light, as discussed below). An exemplary device is shown in FIG. 1. The sensors include patterned growth of myotubes and neurites on the graphene, producing NMJs at defined spatial locations in the array. Each sensor potentially has multiple NMJs (such as 2, 4, 6, 8, 10, or more NMJs). For example, a 2×4 cm array of 60×60 sensors with 10 NMJs per sensor could have up to 36,000 NMJs, or about 4500 NMJs/cm² (about 11,250- to 45,000-fold increase in density over current methods). Even if only 10% of the sensors are functional and only about 20% of the patterned myotubes and neurites form connections (NMJs), this array would have about 720 NMJs, or 90 NMJs/cm². This provides at least about a 225- to 900-fold increase in density over current in vitro methods of generating NMJs.

In addition, in the devices, systems, and methods provided herein, the NMJs are at defined spatial locations and use of patterned graphene (or other materials) allows for simultaneous recording of activity. These features make the disclosed devices suitable for HTS of compounds affecting muscle cell, neuronal cell, and/or NMJ activity.

In some embodiments, the neuronal cells utilized on the device are genetically modified cells expressing a heterologous nucleic acid that allows the cells to be activated by light (referred to in some instances as “optogenetically active” cells), allowing the neuronal cells on the array to be simultaneously activated by exposure to light. In some examples, the neuronal cells express channelrhodopsin-2 (ChR2).

### I. Abbreviations

AchR acetylcholine receptor

ChR2 channelrhodopsin-2

DAPI 4′, 6-diamidino-2-phenylindole

FBS fetal bovine serum

FET field effect transistor

G-FET graphene field effect transistor

HS horse serum

HTS high throughput screening

IGF-1 insulin-like growth factor-1

MEA multi-electrode array

MHC myosin heavy chain

NMJ neuromuscular junction

PDMS polydimethylsiloxane

PMMA poly(methyl methacrylate)

sccm standard cubic centimeter per minute

TUB3 beta III tubulin

VX O-ethyl S-[2-(diisopropylamino)ethyl] methylphosphonothioate

### II. Terms

Unless otherwise noted, technical terms are used according to conventional usage. Definitions of common terms in molecular biology may be found in Krebs et al., Lewin's Genes XI, published by Jones and Bartlett Learning, 2012 (ISBN 1449659853); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 2011 (ISBN 8126531789); and George P. Rédei, Encyclopedic Dictionary of Genetics, Genomics, and Proteomics, 2nd Edition, 2003 (ISBN: 0-471-26821-6).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

In order to facilitate review of the various embodiments of the invention, the following explanations of specific terms are provided:

Electrically active material: A material that is electrically conductive (including semiconductor materials). For example, an electrically active material can have a charge carrier mobility (μ) of about 100-5000 cm²/Vs, an interfacial capacitance (Cᵢₙₜ) of about 0.2-4 μF/cm², and/or transconductance (gₘ/U_{DS}) of about 0.1-6 (mS/V). In some embodiments an electrically active material is graphene or graphene oxide. In other embodiments, an electrically active material is hBN, MoS₂, MoSe₂, MoTe₂, WS₂, silicon, diamond, or AlGaN/GaN. In some non-limiting examples, the electrically active material is biocompatible.

Electrode: An electrical conductor, through which a current enters or leaves a medium (such as a solution, a cell, a NMJ, or an electrically active material). In some examples, an electrode is a recording electrode, such as an electrode which receives current (for example, from a cell or a device disclosed herein). The recording electrode can be connected to an apparatus for visualizing or making a transient, semi-permanent, or permanent record of the received current. In other examples, an electrode is a stimulating electrode, such as an electrode which introduces current to a cell or a device disclosed herein.

Graphene: A two-dimensional sheet of densely packed sp²-bonded carbon atoms arranged in a hexagonal pattern. Graphene conducts heat and electricity (e.g., is electrically active) with high efficiency and is also optically transparent. Graphene is also biocompatible and cell growth and differentiation can be carried out on graphene surfaces.

Myotube: A structure produced by fusion of muscle cells (such as myoblasts) into multi-nucleated fibers. In some examples, myotubes are produced by culturing muscle cells in a differentiation medium, such as a medium containing IGF-1. The disclosed methods may utilize skeletal, cardiac, or smooth muscle cells to form myotubes.

Neurite: A projection from the cell body of a neuron, for example, an axon or a dendrite. In some examples, neurite outgrowth is a marker of differentiation of neuronal cells. NMJs may form where a neurite and a muscle cell (such as a myotube) are in close proximity or physical contact with one another.

Neuromuscular junction (NMJ): A connection (e.g., synapse) between a motor neuron cell and a muscle cell. The NMJ includes an axon terminal of a motor neuron and a motor end plate of a muscle cell. Synaptic vesicles containing acetylcholine (Ach) are present in the presynaptic portion of the neuronal cell and acetylcholine receptors (AchRs) are concentrated on the membrane of the muscle cell at the NMJ. Ach released from the neuron binds to AchRs on the muscle cell and leads to voltage changes in the muscle cell, entry of calcium, and ultimately a muscle action potential. Signaling is terminated by breakdown of Ach in the synaptic cleft by acetylcholinesterase.

Optically activated ion channel: Also referred to as a light-gated ion channel. An ion channel (such as a cation channel) that opens or closes in response to light. Optically activated ion channels include naturally occurring ion channels, such as channelrhodopsins (see, e.g., Nagel et al., Science 296:2395-2398, 2002; Nagel et al., Proc. Natl. Acad. Sci. USA 100:13940-13945, 2003). Synthetic optically activated ion channels have also been engineered, for example by modification of voltage-gated or other types of ion channels (see, e.g., Banghart et al., Biochemistry 45:15129-15141, 2006).

An example of an optically-activated (light-gated) cation channel is channelrhodopsin-2 (ChR2), which is an ion channel derived from algae (Nagel et al., Proc. Natl. Acad. Sci. USA 100:13940-13945, 2003). The channel opens upon absorption of a photon and is permeable to monovalent and divalent cations. Thus, exposure of a cell expressing ChR2 to light results in depolarization of the cell. In some examples, cell expressing heterologous ChR2 (for example, mammalian cells) are referred to as “optogenetically active” cells.

Substrate: A solid surface capable of supporting one or more components, such as sensors, cells, and/or electrodes. In some examples, a substrate includes a substantially flat surface comprising silicon/silicon dioxide or glass. In other examples, a substrate comprises a polymer or hydrogel (such as PDMS).

Toxin: A compound or substance that is poisonous or harmful to cells or living organisms. In some examples, toxins include neurotoxins and/or nerve agents, which are compounds or substances that are poisonous to, or destructive of, neuronal cells or tissue. Exemplary neurotoxins include but are not limited to naturally occurring toxins (such as tetrodotoxin, conotoxin, botulinum toxin, tetanus toxin, bungaratoxin, curare, and chlorotoxin) and synthesized nerve agents (such as sarin, VX, soman, tabun, and other organophosphorus compounds).

### III. Device and System Embodiments

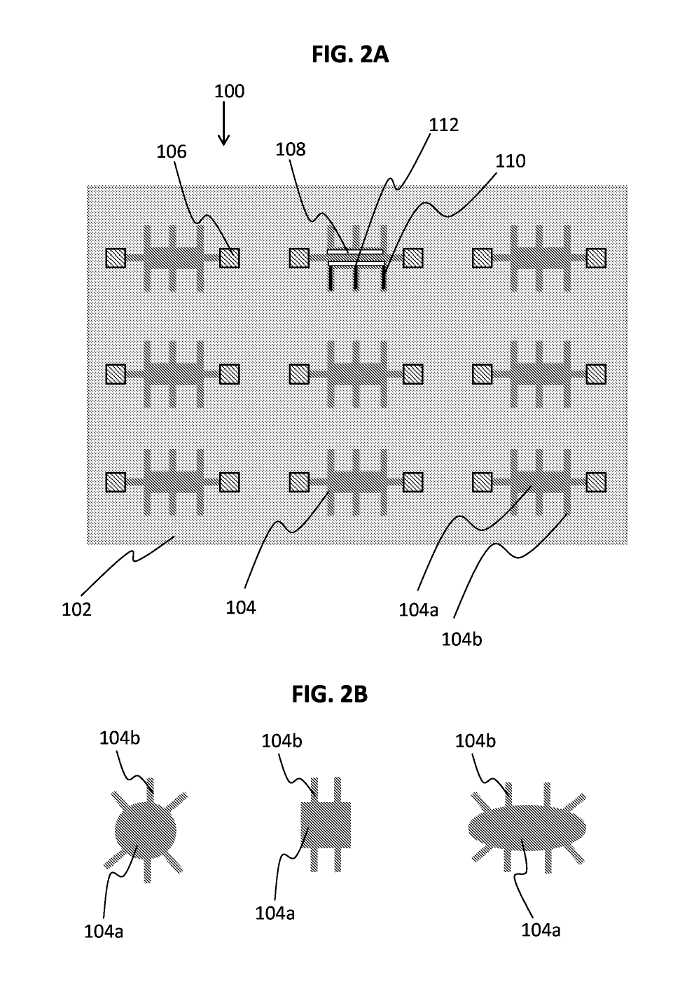

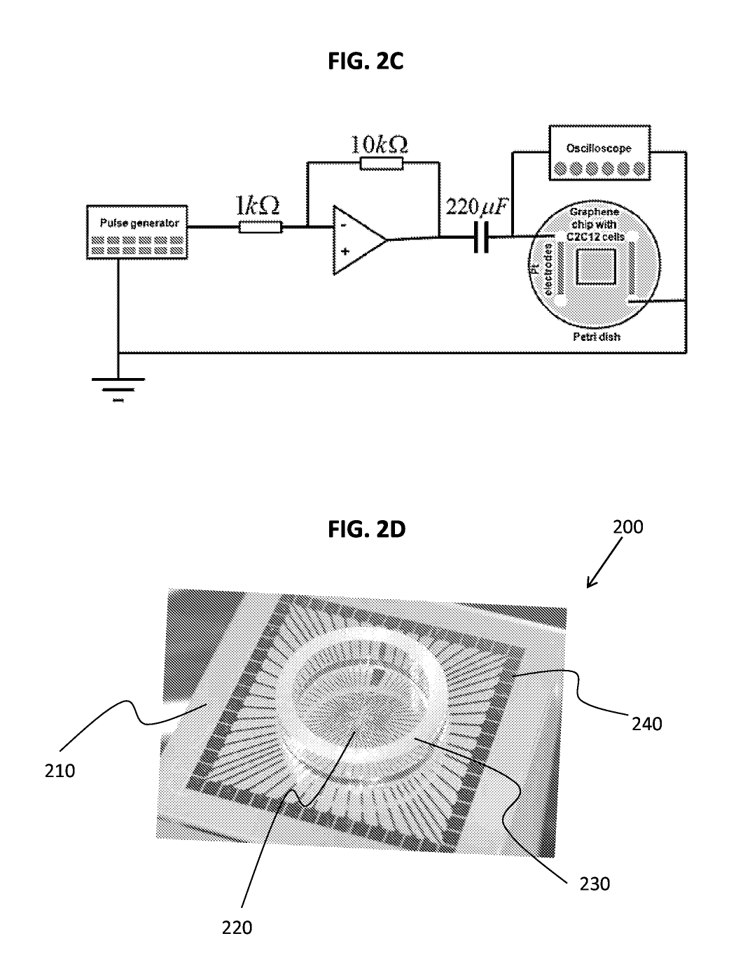

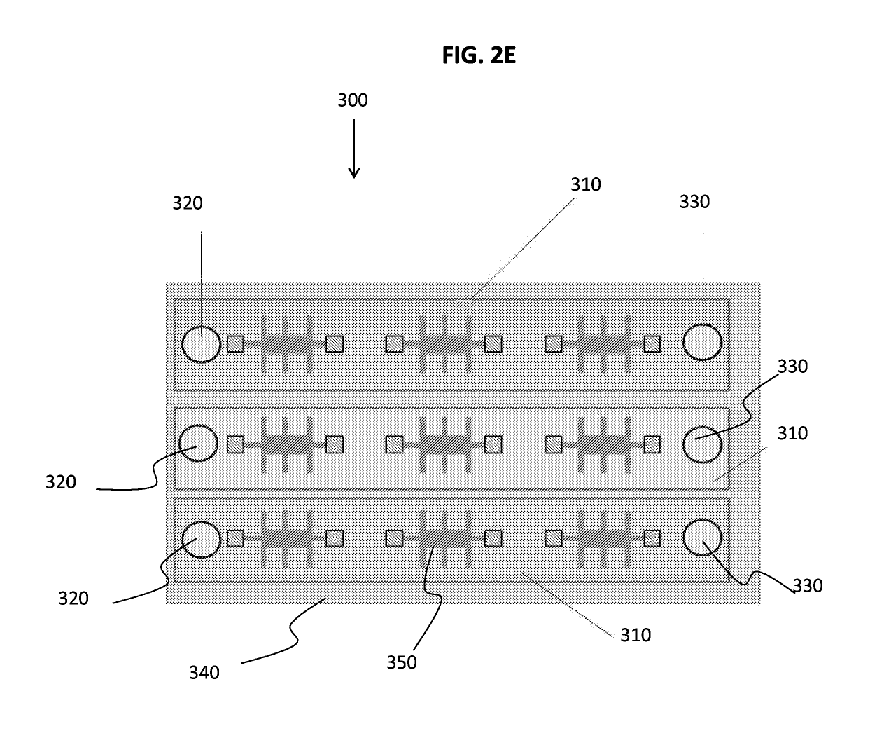

Disclosed herein are devices that include one or more regions of electrically active material (such as graphene) disposed upon a substrate (such as silicon dioxide), wherein the electrically active material is patterned to accommodate formation of one or more NMJs at a defined location. The devices optionally also include one or more electrodes (such as one or more stimulating and/or recording electrodes), and optionally one or more electrical circuits, such as a stimulatory circuit and/or a recording circuit. In some embodiments (such as in systems disclosed herein), the regions of electrically active material include cells (such as muscle cells and neuronal cells) that form NMJs, for example at defined locations, based on the patterning (shape or arrangement) of the electrically active material. Exemplary embodiments of the device (or components thereof) are shown in FIGS. 1 and 2A-2E (discussed in more detail below).

The devices disclosed herein include a substrate, upon which the other components of the device (such as the electrically active material, cells, and/or electrodes) are disposed (for example, in physical contact with the substrate). The substrate is a solid support for the components that provides structural integrity for the device, for example for manipulation and/or transport of the device or its components. In some examples, the substrate includes silicon or silicon dioxide (SiO₂) or glass. In other examples, the substrate is a polymer, such as polydimethylsiloxane. The substrate can be any size suitable for accommodating the desired number and size of regions of electrically active material patterned on the substrate. In some examples, the substrate is about 0.5 to about 10 cm², for example about 5 to about 10 cm², about 3 to about 6 cm², or about 1 to about 2 cm². In one particular example, the substrate is about 8 cm² (for example, about 2 cm×4 cm). However, one of skill in the art will appreciate that smaller or larger substrates can also be used in the devices and methods disclosed herein.

As discussed above, the device includes one or more (such as a plurality of) regions of electrically active material (also referred to herein as “islands” or “sensors”) disposed on the substrate. The electrically active material can be any material that is electrically conductive (including semiconductor materials). In some non-limiting examples, an electrically active material can have a charge carrier mobility (u) of about 100-5000 cm²/Vs, an interfacial capacitance (Cᵢₙₜ) of about 0.2-4 μF/cm², and/or transconductance (gₘ/U_{DS}) of about 0.1-6 (mS/V). In particular examples, the electrically active material is also biocompatible (for example, does not inhibit cell growth or formation of NMJs) and/or stable in an aqueous environment (for example, in cell culture media). Optionally, the electrically active material is also optically transparent. Optical transparency is advantageous both for imaging (for example, either inverted or upright microscope can be used for imaging) and/or if optogenetically active cells are utilized. In some embodiments an electrically active material is graphene or graphene oxide. In other embodiments, an electrically active material is hBN, MoS₂, MoSe₂, MoTe₂, WS₂, silicon, diamond, or AlGaN/GaN.

In some embodiments, the one or more regions of electrically active material disposed on the substrate include one or more layers of the electrically active material (for example, one or more layers of graphene). Single layer thicknesses of electrically active material are advantageous because they have the highest transconductance and carrier mobility and are thus electrically most conductive. However, sensors with more than one layer (such as two, three, four, five, or more layers) of electrically active material are also electrically conductive and can be used for the devices and methods described herein. In some examples, the number of layers of electrically active material in a region on the substrate is homogeneous, while in other examples, the number of layers of electrically active material in a region on the substrate is heterogeneous. In one example, a region is primarily a single layer of electrically active material, but may have two or three layers of electrically active material in at least a portion of the region. In other examples, all or substantially all of the region of electrically active material is one layer thick.

In some embodiments, a region of electrically active material on the substrate (also referred to as a “sensor”) is patterned (for example, has a particular shape and/or orientation) to facilitate formation of NMJs at particular defined locations or areas of the material. This can assist with identification of location of NMJs on the device, as well as with facilitating recording and/or analysis of NMJ activity. The sensor can have any shape, including square, rectangular, round, oval, or an irregular or multi-part shape. As discussed below, a region of electrically active material can include two or more areas having different shapes and/or orientations to facilitate cell growth or formation of NMJs at defined locations.

In some examples, one or more of the sensors include at least two areas (such as 2, 3, 4, 5, 6, 7, 8, 9, 10, or more areas, for example 2-50, 2-20, 5-30, or 7-11 areas). Two or more of the areas are in physical contact, but have different shapes, dimensions, and/or orientations. In some examples, the muscle cells (or myotubes) are present in a first area of the sensor and the neuronal cells are present in a second area of the sensor. NMJs are expected to form at the location(s) where the first and second areas meet or are adjacent, for example, where the muscle and neuronal cells are in close proximity or contact. In some examples, a sensor includes a first central area (which includes muscle cells or myotubes in some examples) and at least one second area that is perpendicular to and in contact with the first central area (which includes one or more neuronal cells or processes in some examples). In particular examples, the sensor includes a rectangular central first area and two or more (for example, 4, 6, 8, 10, 12, or more) second areas that are oriented perpendicular to the first area and are in direct contact with the first area. Some exemplary arrangements are shown in FIGS. 1 and 2A. In other examples, the at least one second area (or third, fourth, fifth, sixth area, and so on, if present) is oriented radially to and in contact with the first area, without necessarily being perpendicular to the first area. Exemplary additional sensor shapes and arrangements are illustrated in FIG. 2B.

The region(s) of electrically active material (the sensors) can be of any size suitable for the size of the substrate. In examples where the shape of the sensor is roughly rectangular (for example, as shown in FIGS. 1 and 2A), the sensor is about 100 μm to 10 mm long (such as about 200 μm to 1 mm long) by about 10 μm to 1 mm wide (such as about 50 μm to 500 μm wide). In some examples, the sensor is about 40-200 μm wide and about 100-500 μm long. In one particular example, the sensor is about 300 μm long by about 100 μm wide. In one example, where the shape of the sensor is roughly circular (e.g., as shown in FIG. 2B), the overall diameter of the sensor is about 10 μm to 10 mm (such as about 100 μm to 1 mm, or about 250 μm to 500 μm).

In particular embodiments, the device includes an array of sensors. For example, the device includes two or more regions of electrically active material that are not in contact with one another. An array of sensors includes an arrangement of sensors in addressable locations on a substrate. In some examples herein, particularly when the device includes an array of sensors, the device is referred to as a “chip.” An array of sensors on the substrate makes it possible to carry out a very large number of analyses (for example, analysis of activity of multiple NMJs) at one time.

Within an array, each sensor is addressable, in that its location can be reliably and consistently determined within at least two dimensions of the array. The sensor location on an array can assume different shapes. For example, the array of sensors can be regular (such as arranged in uniform rows and columns) or irregular. In particular examples disclosed herein, the sensors are arranged in a symmetrical grid pattern on the substrate, but sensors could be arranged in other patterns (such as in radially distributed lines, spiral lines, or ordered clusters). Addressable arrays usually are computer readable, in that a computer can be programmed to correlate a particular address on the array with information about the sensor at that position (such as electrical activity). In some examples of computer readable formats, the individual sensors are arranged regularly, for instance in a Cartesian grid pattern, which can be correlated to address information by a computer. Exemplary devices including an array of regions of electrically active material (sensors) are shown in FIGS. 1 and 2A.

In some embodiments, the device includes an array of two or more sensors disposed on the substrate, for example, at least 2, 3, 4, 5, 10, 12, 24, 36, 48, 60, 72, 96, 500, 1000, 1500, 2000, 3000, 4000, 5000, 10,000, 20,000, 50,000, or more sensors, such as 2-3600, 10-3000, 1000-5000, 1000-4000, 4000-10,000, or 20,000-50,000 sensors. In one particular example, the device includes 3600 sensors, for example arranged as a 60×60 grid of sensors. The number of sensors in the device will be at least partly determined by the dimensions of the substrate, as well as the size of the sensors themselves and the computing power of any associated recording system. In addition, the number of sensors will be determined by the spacing between the sensors. In some examples, the sensors are separated from one another by at least about 5 μm, 10 μm, 25 μm, 50 μm, 100 μm, 150 μm, or more (such as about 10-500 μm, 50-250 μm, or 100-200 μm). In one non-limiting embodiment, the sensors are separated from one another by about 100 μm in each dimension. One of skill in the art can select the number of sensors for a substrate of a given size, depending on the size of the sensors and the size of the substrate. In some embodiments, all of the sensors on an array are of the same shape, arrangement, and/or dimensions. In other embodiments, two or more of the sensors on the array have a different shape, arrangement, and/or dimension from one another.

In some embodiments, one or more of the sensors on the substrate are coupled to (for example, electrically connected to) one or more electrodes, such as stimulation and/or recording electrodes. In some examples, the one or more electrodes are coupled to the first area of the sensor (e.g., the area including the muscle cells (or myotubes)). In other examples, the one or more electrodes are coupled to the second area of the sensor (e.g., the area including the neuronal cells or neuronal processes). In further examples, the one or more electrodes are coupled to the portion of the sensor at or near where the first area and the second area are in contact (such as the area including one or more NMJs). The electrode(s) can be of any suitable material and can be selected by one of skill in the art. In some examples, the electrode(s) include gold, platinum, titanium (e.g. titanium oxide or titanium nitride), iridium (e.g., iridium oxide), or combinations thereof. In some examples, a layer of platinum or titanium is photolithographically patterned, followed by evaporation of gold to prepare the electrode.

In some embodiments, the one or more electrodes are coupled to circuitry which generates an extracellular stimulus (e.g., a stimulus that induces an action potential in a neuronal cell or a contraction in a muscle cell), referred to herein as stimulation circuitry. In other embodiments, the one or more electrodes are coupled to circuitry for detecting and/or recording changes in the electrical activity (e.g., activity of a neuronal cell or a muscle cell), referred to herein as recording circuitry. In some examples, the one or more electrodes are coupled to stimulation circuitry and recording circuitry. The circuitry may include one or more of a current generator, amplifier, capacitor, and/or an oscilloscope. An exemplary electrical circuit is illustrated in FIG. 2C. One of skill in the art can select appropriate stimulation and/or recording circuitry for use with the disclosed devices and systems.

In some embodiments, the electrically active material comprises a field effect transistor (FET) (such as a graphene FET (G-FET), if the electrically active material is graphene). See, e.g., Dankerl et al., Advanced Functional Materials, 2010, 20:3117-3124; Choi et al., Biomedical Engineering Letters, 2013, 3:201-208; Cheng et al., Nano Letters, 2013, 13:2902-2907; Hess et al., Proceedings of the IEEE, 2013, 101:1780-1792; Hess et al. Applied Physics Letters, 2011, 99:033503; He et al., ACS Nano, 2010, 4:3201-3208; Hess et al., Adv. Mater. 2011, 23:5045-5049; Chen et al., Physica E: Low-dimensional Systems and Nanostructures, 2007, 40:228-232; Han et al., Phys. Rev. Lett., 2007, 98:206805; Feng et al., Nanoscale, 2012, 4:4883-4899; Cabruja et al., Surface Science, 1991, 251-252:364-368; Cohen-Karni et al., Nano Letters, 2010, 10:1098-1102. An FET uses an electric field to control the conductivity of the channel. An advantage of an FET is the gain (amplification) of the signal. For example, for a very small input change, a very large change in the output is produced which helps to measure even very minute differences in the system.

FIG. 2A illustrates an exemplary embodiment of a disclosed device 100. As illustrated in FIG. 2A, regions of electrically active material 104 are disposed on substrate 102. The regions of electrically active material 104 include a first central area 104a in contact with perpendicularly oriented second areas 104b. The regions of electrically active material 104 are each connected to at least one electrode 106. In some embodiments, muscle cells (for example, myotubes) 108 are on the first central area 104a of the electrically active material 104 and neuronal cells (such as neuronal cells with neurites) 110 are on the second areas 104b. In some examples, a neuronal cell 110 forms a NMJ 112 with a muscle cell 108. In other embodiments, neuronal cells (for example, neuronal cells with neurites) 108 are on the first central area 104a of the electrically active material 104 and muscle cells (such as myotubes) 110 are on the second areas 104b. In this example, a neuronal cell 108 forms a NMJ 112 with a muscle cell 110. While FIG. 2A illustrates a particular configuration of the electrically active material, electrodes, and cells, any suitable configuration can be used. Additional configurations of the electrically active material are shown in FIG. 2B, each having a first central region 104a and a second region 104b in contact with and oriented perpendicularly (or substantially perpendicularly) to the central area or at an angle of at least 5° with respect to the first area 104a.

In some embodiments, the circuitry of an array disclosed herein is (or is similar to) a multi-electrode array (MEA) chip. For example, as illustrated in FIG. 2D, a MEA chip 200 includes a substrate (or support) 210, a central portion having multiple graphene FETs 220 where the cells are cultured within an enclosure 230 and multiple electrodes 240 connecting to the graphene portion including the cells. The FET 230 can be addressed through the outer connections (electrodes). Although each of the electrodes is individually addressable, circuits similar to the one for MEA recording can be used to obtain signals for multiple FETs simultaneously. The MEA can be housed in an external measurement system and signals can be acquired in real-time.

In some embodiments, the device includes channels or other means for providing fluids (such as media and/or test compounds) to the cells. Thus, in some examples, the device includes microfluidics, for example, so that each electrically active region or a subset of electrically active regions (for example, a row or column of regions in an array) can be exposed to different conditions, such as different compounds and/or concentrations of compounds. An exemplary device, including microfluidic channels is shown in FIG. 2E. The exemplary device 300 includes three microfluidic channels 310, each with an inlet 320 and an outlet 330, which can be used to introduce and remove fluids (including test compounds, if desired), respectively. The inlet(s) and/or outlet(s) may be attached to additional components for introducing or removing fluids, such as tubing, syringes, pumps, and so on (not shown). Each channel is attached to an underlying substrate 340, which includes one or more electrically active regions 350 (such as those shown in FIG. 2A).

In some embodiments, the device optionally further includes a container (such as a reservoir, dish or compartment) capable of holding fluid. The device (for example, the substrate and components disposed on the substrate) is placed in or on the container and submerged in a fluid (such as a culture medium).

Also disclosed herein are systems, which in some embodiments are a disclosed device that includes cells capable of forming NMJs, which are present on the region(s) of electrically active material on the substrate. In some examples, the cells include muscle cells (which in some examples are differentiated muscle cells, such as myoblasts or myotubes) and neuronal cells (which in some examples are differentiated neuronal cells, such as neuronal cells with neuronal processes or neurites). In some examples, the muscle cells (or myotubes) are present in a first area of the electrically active region and the neuronal cells are present in a second area of the electrically active region. NMJs are expected to form at the location(s) where the first and second areas meet or are adjacent, for example, where the muscle and neuronal cells are in close proximity or contact with one another. In some examples, a disclosed system includes one or more muscle cells or myotubes on a first area of an electrically active region and includes one or more neuronal cells or processes on a second area of an electrically active region. In other examples, a disclosed system includes one or more neuronal cells or processes on a first area of an electrically active region and includes one or more muscle cells or myotubes on a second area of an electrically active region.

In particular examples, the muscle cells include skeletal muscle cell lines or primary skeletal muscle cells (for example, primary human skeletal muscle cells). An exemplary skeletal muscle cell line is C2C12 mouse skeletal muscle cells. Other skeletal muscle cell lines that could be used in embodiments of the device include L6 cells (rat), Sol8 cells (mouse), or G-7 cells (mouse). In other examples, the muscle cells include stem cells (such as embryonic stem cells, pluripotent stem cells, or induced pluripotent stem cells), which are differentiated to muscle-like cells or myotubes. In particular examples, the neuronal cells include motor neuron cell lines or primary motor neuron cells. Exemplary motor neuron cell lines include mouse NSC-34 cells and mouse HB9 cells (Aruna Biomedical, Inc., Athens, Ga.). In other examples, the neuronal cells include stem cells (such as embryonic stem cells, pluripotent stem cells, or induced pluripotent stem cells), which are differentiated to neuron-like cells or cells with neural processes or neurites. Muscle and neuronal cells are available from commercial sources, including American Type Culture Collection (ATCC, Manassas, Va.), Cellutions Biosystems, Inc. (Burlington, Ontario, Canada), Sigma-Aldrich (St. Louis, Mo.), and other sources. One of skill in the art can select additional cells or cell lines for the devices disclosed herein.

In some embodiments, one or more of the cells included in the device (such as the muscle cells or the neuronal cells) are genetically modified to include an optically active protein that can be used to alter the activity of the cell(s). In some examples, these cells are referred to as “optogenetically” modified or engineered cells. In some examples, the cells included in the device express channelrhodopsin-2 (ChR2), also referred to as chlamyopsin 4 light-gated ion channel. ChR2 is an optically activated cation channel derived from algae (e.g., Chlamydomonas reinhardtii). Thus, it can be used to non-invasively control activity of a single cell or a population of cells (such as neuronal cells or muscle cells) expressing the protein using light stimulation. ChR2 nucleic acid and amino acid sequences of use to produce the disclosed optogenetically modified cells include GenBank Accession Nos. AF461397 and XM_001701673 (nucleic acid) and AAM15777 and XP_001701725 (protein), all of which are incorporated herein by reference as present in GenBank on Nov. 14, 2015. One of skill in the art can identify other ChR2 nucleic acid and amino acid sequences suitable for use in the disclosed devices and methods.

Methods of modifying cells to express ChR2 are known to one of skill in the art and include transforming cells with a viral vector encoding the protein (such as a lentivirus vector) or a plasmid with the protein operably linked to a regulatable or cell-type specific promoter. Methods of optogenetically modifying cells and regulating their activity are described in Boyden et al., Nature Neurosci. 8:1263-1268, 2005; Bruegmann et al., Nature Meth. 7:897-900, 2010; Deisseroth et al., Ann. Rev. Neurosci., 2011; and Sakar et al., Lab Chip 12:4976-4985, 2012, all of which are incorporated by reference herein.

### IV. Methods of Making the Devices and Systems

Disclosed herein are methods for making the devices and systems described in Section III. In some embodiments, the methods include making a substrate with one or more patterned regions of electrically active material and optionally including one or more electrodes and/or microfluidic channels. In other embodiments, the methods include making a substrate with one or more patterned regions of electrically active material and optionally including one or more electrodes and/or microfluidic channels, followed by introducing neuronal and muscle cells (or myotubes) to one or more of the patterned regions and forming NMJs at one or more defined locations. In other embodiments, the methods include introducing neuronal and muscle cells to a pre-made substrate with patterned regions of electrically active material and forming NMJs at one or more locations.

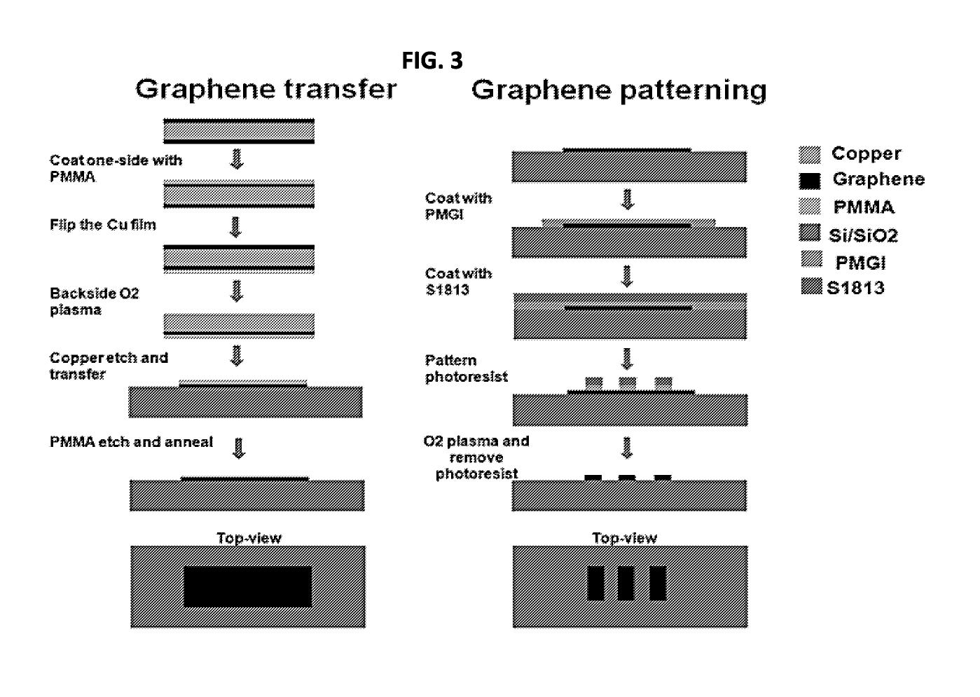

The electrically active material on the substrate is patterned to produce the desired arrangement and shape(s) of regions of the electrically active material, as discussed above. Patterning of electrically active material on a substrate can be performed by standard techniques, such as photolithography. In a particular example, an electrically active material (such as graphene) is patterned on a substrate (such as Si/SiO₂) as described in Bajaj et al. (Adv. Healthcare Mater. 3:995-1000, 2014; incorporated by reference herein in its entirety) and illustrated in FIG. 3. Briefly, graphene is grown using chemical vapor deposition on a copper foil. Exemplary growth parameters are CH₄:H₂:Ar=100:50:1000 sccm (standard cubic centimeter per minute) at 1000° C., growth time 40 minutes, growth pressure 100 mTorr. One side of the copper foil (with graphene) is coated with a polymer (for example, poly(methyl methacrylate) (PMMA)) and the other side is etched by oxygen plasma. The copper is then etched using FeCl₃ and the graphene is transferred onto the substrate. The PMMA is etched using dicholoromethane:methanol solution and annealed. The desired graphene pattern on the substrate is produced using standard photolithography techniques. One of skill in the art can identify suitable methods for growth and patterning of other electrically active materials, such as graphene oxide, hBN, MoS₂, MoSe₂, MoTe₂, and WS₂.

Additional patterning methods are known to one of skill in the art and can be used to produce the patterned electrically active material on a substrate included in the devices described herein. For example, the patterning can be produced using electron-beam or ultraviolet lithography followed by a lift-off process (e.g., Ye et al., Nanoscale 3:1477-1481, 2011), soft lithographic techniques (e.g., Jung et al., Nanotechnology 25:285-302, 2014), and laser-induced pattern transfer (Yoo et al., Small 9:4239-4275, 2013). Additional patterning techniques are described in Feng et al. (Nanoscale 4:4883-4899, 2012).

The regions of electrically active material are connected to one or more electrodes, which are disposed on the substrate. In some embodiments, the electrodes are also connected to one or more electrical circuits, such as electrical stimulation and/or recording circuits. In some embodiments, the regions of electrically active material are connected to standard electrical circuitry such as micromanipulator probes and a testing system for signal recording (such as systems available from Keithley Instruments, Cleveland, Ohio).

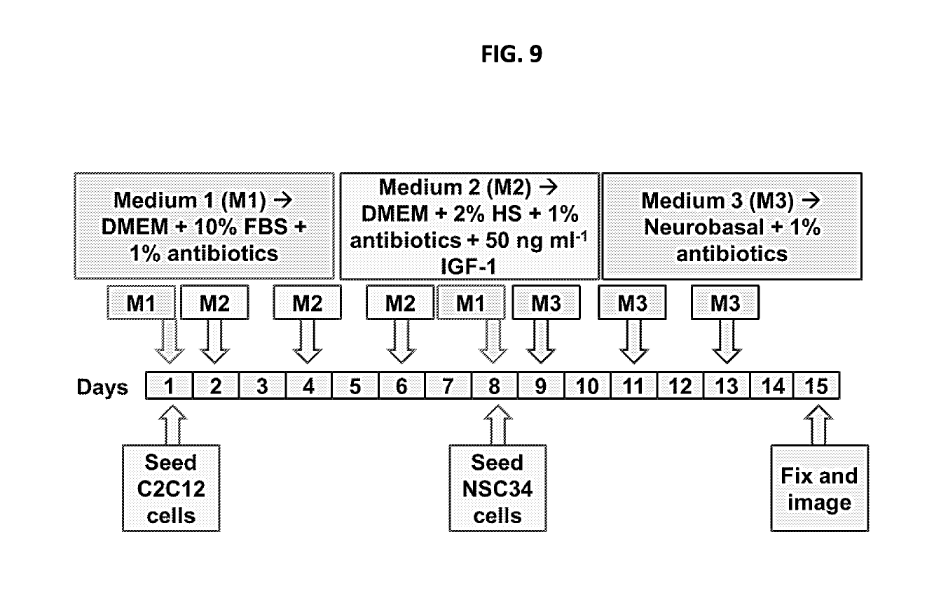

In some embodiments, the methods of producing the disclosed devices include introducing muscle cells and neuronal cells to the region(s) of electrically active material patterned on the substrate. In some examples, the cells are seeded in wells that each include a patterned region of electrically active material. The well can be made of any suitable material, for example, polymers such as polydimethylsiloxane (PDMS). In some examples, the muscle cells and neuronal cells are introduced sequentially, while in other examples, the muscle cells and neuronal cells are introduced simultaneously or substantially simultaneously. An exemplary non-limiting protocol for seeding and differentiating the muscle and neuronal cells is shown in FIG. 9. In some examples, it is advantageous to seed and differentiate muscle cells first, as it is believed that the thin graphene regions (e.g., 104b of FIG. 2A) reduce or prevent myotube formation on them (see, e.g., Bajaj et al., Integr. Biol. 3:897-909, 2011) and this design may encourage formation of highly patterned NMJ over the device.

In one example, muscle cells (e.g., myoblasts, such as C2C12 cells) are seeded on the electrically active material (or a specific area of the electrically active material). It is expected that the cells will attach everywhere on the device, but that myotubes will only form on the “thicker” regions of the electrically active material (e.g., 104a of FIG. 2A). See e.g., Bajaj et al., Integr. Biol. 3:897-909, 2011. The muscle cells are cultured in some examples for about 1-10 days (such as 1, 2, 3, 4, 5, 6, 7, 8, 9, or 10 days) in a growth medium. In other examples, the cells are cultured until they reach about 80-90% confluence. After a period of time or when the desired level of confluence is reached, the growth medium is replaced with differentiation medium to enhance differentiation of the myoblasts to myotubes. In some examples, the differentiation medium includes insulin-like growth factor 1 (IGF-1, for example, about 1 ng/ml to 500 ng/ml, such as about 10 ng/ml to 100 ng/ml, or 50 ng/ml IGF-1). The myoblasts are cultured in differentiation medium for a sufficient period of time for development of myotubes, for example about 1 day to 14 days (such as about 2-10 days, 6-10 days, or 6-7 days). In some examples, the myoblasts are cultured in the differentiation medium until a desired percentage of myotubes is formed (such as 10 to 90% or 30 to 70% myotube formation). In other examples, a number of days (such as 10 days or less) is selected to avoid contraction and/or detachment of the myotubes from the electrically active material. In some examples, it is believed that use of a softer substrate than SiO₂ (such as PDMS or other hydrogels) could reduce this effect.

Following differentiation of myotubes, neuronal cells (e.g., motor neuron cells, such as NSC-34 cells) are seeded on the device. It is expected that the neurons will attach everywhere on the device, but the neurites prefer to grow along the thinner graphene “lanes” (e.g. 104b of FIG. 2A), particularly, as the thicker graphene portions (e.g., 104a of FIG. 2A) are occupied by the muscle cells (myotubes), and hence is believed to lead to NMJ formation in a controlled and high-throughput fashion. Growth of neuronal cells on patterned graphene is described, e.g., Lorenzoni et al., Sci. Rep. 3:1954, 2013; Bendali et al., Adv. Healthcare Mat. 2:929-933, 2013. In particular examples, the neuronal cells are modified to express a light-sensitive protein, such as channelrhodopsin-2 (discussed above).

The myotubes and neuronal cells are cultured in the original growth medium for about 1-6 days (such as 1, 2, 3, 4, 5, 6, or more days). After a period of time (such as 1 day in the example shown in FIG. 9), the growth medium is replaced with a neuronal differentiation medium to enhance differentiation of the neuronal cells (e.g., formation of neurites). In some examples, the neuronal differentiation medium can include Neurobasal medium (e.g., available from Life Technologies, Grand Island, N.Y.), Neurobasal medium with addition of N2 supplement, B27 supplement and/or IGF-1, or DMEM/F12 (1:1) medium, optionally with 1% NEAA and/or 1 μM retinoic acid. In some examples, agrin is included in the neural differentiation medium (for example, about 100-500 ng/ml agrin) to increase clustering of AchRs and NMJ formation. The cells are then cultured in the differentiation medium for a sufficient period of time for neurites to form and/or for development of NMJs between neurites and myotubes, for example about 1 day to 14 days (such as about 2-10 days, 2-8 days, or 5-6 days).

### V. Methods for Using the Devices and Systems

Disclosed herein are methods of using the disclosed devices or systems. Particular method embodiments disclosed herein include introducing one or more compounds (for example, a drug, toxin, stimulus, or composition thereof), to a device or system embodiment disclosed herein and analyzing a response generated by the device after introducing the compound(s). In particular examples, the methods include contacting one or more cells (or populations of cells) and/or the NMJs present in the device with one or more compounds and analyzing a response of one or more cells (or populations of cells) in the device. In some embodiments, the devices disclosed herein can be used in combination with cells from a subject with a disease that affects nerve or muscle function (such as NMJ function) and can be used to screen for compounds with potential therapeutic activity. In other examples, the devices disclosed herein could be seeded only with muscle cells (or myotubes) and contacted with one or more compounds to identify compounds that affect electrical activity or contractility of muscle cells independently of neuronal cells or seeded only with neuronal cells and contacted with one or more compounds to identify compounds that affect neuronal cell electrical activity independently of muscle cells. In such embodiments, the regions of electrically active material may be patterned to include only the first area (e.g., 104a of FIG. 2A).

In some examples, the methods further include stimulating (such as evoking an activity, such as electrical activity) one or more cells or populations of cells (such as one or more neuronal cells or a population of neuronal cells) before, after, or simultaneously with introducing the compound(s). In some examples, the stimulation is from direct electrical stimulation (for example, introducing an electrical current to the neuronal cell(s)). In other examples, the stimulation is indirect, for example, by exposing neuronal cells expressing an optically active ion channel (such as channelrhodopsin-2) to a light stimulus.

In some embodiments, analyzing a response includes detecting a change in activity of one or more of the neuronal cells and/or muscle cells compared to a control (such as untreated cells or NMJs, or cells or NMJs treated with a compound having a known effect, such as a toxin) or a reference value. In some examples, the activity includes electrical activity. In other examples, the activity includes a change in contractility of the muscle cells. A compound that increases or decreases activity can be identified as a potential toxin or therapeutic compound and can be selected for further analysis (such as in vitro or in vivo testing).

In one particular embodiment, the methods include screening for a counter-toxin to a toxin that affects contractility of muscle cells. For example, the method includes contacting NMJs present in the device with a toxin and measuring contractility of the muscle cells before and after addition of a counter-toxin or other test compound (such as a candidate counter-toxin). For example, some chemical warfare agents block acetylcholinesterase and cause the muscle to contract vigorously or remain contracted. By addition of a counter-toxin, this contraction is reduced or inhibited. In one particular example, from a signaling point of view (electrical signal acquisition) when the toxin is effective random noise will be observed, while if the cells are contracting naturally (for example, after addition of a counter-toxin) a sinusoidal signal will be observed. Particular toxins that can be used in this exemplary assay include sarin, soman, and VX, as well as pesticides or insecticides known to affect the NMJ (such as organophosphate toxins).

A “compound” or “test compound” is any substance or any combination of substances that is useful for achieving an end or result. The compounds analyzed using the methods disclosed herein can be of use for modulating activity of a NMJ or neuronal or muscle cell activity or function. Exemplary compounds include toxins or potential toxins (e.g., neurotoxins), including organophosphorus compounds. Additional exemplary compounds include therapeutic compounds or candidate therapeutic compounds, such as countertoxins or compounds effective for ameliorating or inhibiting symptoms of a NMJ-associated disease.

Exemplary compounds include, but are not limited to, peptides, such as soluble peptides, including but not limited to members of random peptide libraries (see, e.g., Lam et al., Nature, 354:82-84, 1991; Houghten et al., Nature, 354:84-86, 1991), and combinatorial chemistry-derived molecular libraries made of D-and/or L-configuration amino acids, phosphopeptides (including, but not limited to, members of random or partially degenerate, directed phosphopeptide libraries; see, e.g., Songyang et al., Cell, 72:767-778, 1993), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), small organic or inorganic molecules (such as, so-called natural products or members of chemical combinatorial libraries), molecular complexes (such as protein complexes), or nucleic acids (such as antisense compounds).

Appropriate compounds can be contained in libraries, for example, synthetic or natural compounds in a combinatorial library. Numerous libraries are commercially available or can be readily produced; means for random and directed synthesis of a wide variety of organic compounds and biomolecules, including expression of randomized oligonucleotides, such as antisense oligonucleotides and oligopeptides, also are known. Alternatively, libraries of natural compounds in the form of bacterial, fungal, plant and animal extracts are available or can be readily produced. Additionally, natural or synthetically produced libraries and compounds are readily modified through conventional chemical, physical and biochemical means, and may be used to produce combinatorial libraries. Such libraries are useful for the screening of a large number of different compounds. Exemplary libraries are available from the NIH Molecular Libraries Program (Molecular Libraries Small Molecule Repository), the NIH Developmental Therapeutics Program compound sets, GlaxoSmithKline, Sigma-Aldrich, Microsource Discovery Systems, ChemBridge, SelleckChem, DNA2.0, AbCheck, GenScript, Thermo Fisher Scientific, GE Dharmacon, Cellecta, Charles River, Phoenix Pharmaceuticals, the EPA ToxCast™ library, and the World Toxin Bank. One of ordinary skill in the art can identify suitable compounds and/or libraries for use in the methods disclosed herein.

In one embodiment, high throughput screening methods involve providing a combinatorial chemical or peptide library containing a large number of potential toxins and/or therapeutic compounds. Such combinatorial libraries are then screened in one or more assays as described herein to identify those library members (particularly chemical species or subclasses) that display a desired characteristic activity (altering NMJ activity). The compounds identified using the methods disclosed herein can serve as conventional “lead compounds” or can themselves be used as potential or actual therapeutics.

The following examples are provided to illustrate certain particular features and/or embodiments. These examples should not be construed to limit the disclosure to the particular features or embodiments described.

### EXAMPLE 1

### Differentiation of C2C12 Myoblasts and Growth on Graphene

This example describes differentiation of C2C12 myoblasts to myotubes in vitro. The data in this example have been previously published (Bajaj et al., Adv. Healthcare Mat. 3:995-1000, 2014 (e-published Dec. 18, 2013), which publication is incorporated herein by reference in its entirety).

C2C12 skeletal myoblasts (ATCC Cat. No. CRL 1772) were cultured on graphene films or SiO₂ in high glucose DMEM supplemented with 10% fetal bovine serum (FBS) and 1% antibiotics. To differentiate myoblasts to myotubes, media was switched to high glucose DMEM with 2% horse serum and 1% antibiotics (differentiation medium) with or without 50 ng/ml insulin-like growth factor-1 (IGF-1). Cells were cultured for 5 days and stained with primary antibody MF20 (myosin heavy chain, MHC) overnight at 4° C., followed by goat anti-mouse secondary antibody and the nuclear stain 4′, 6-diamidino-2-phenylindole (DAPI) for 2 hours at 37° C. Cells were imaged with a fluorescent microscope and images were quantified using ImageJ 1.47d.

Fusion index was calculated as the ratio of the number of nuclei in myocytes with two or more nuclei versus the total number of nuclei. Cell density was determined by counting the number of nuclei in a particular area. Statistical analysis was performed using one-way analysis of variance and values are reported as mean±standard error of the mean.

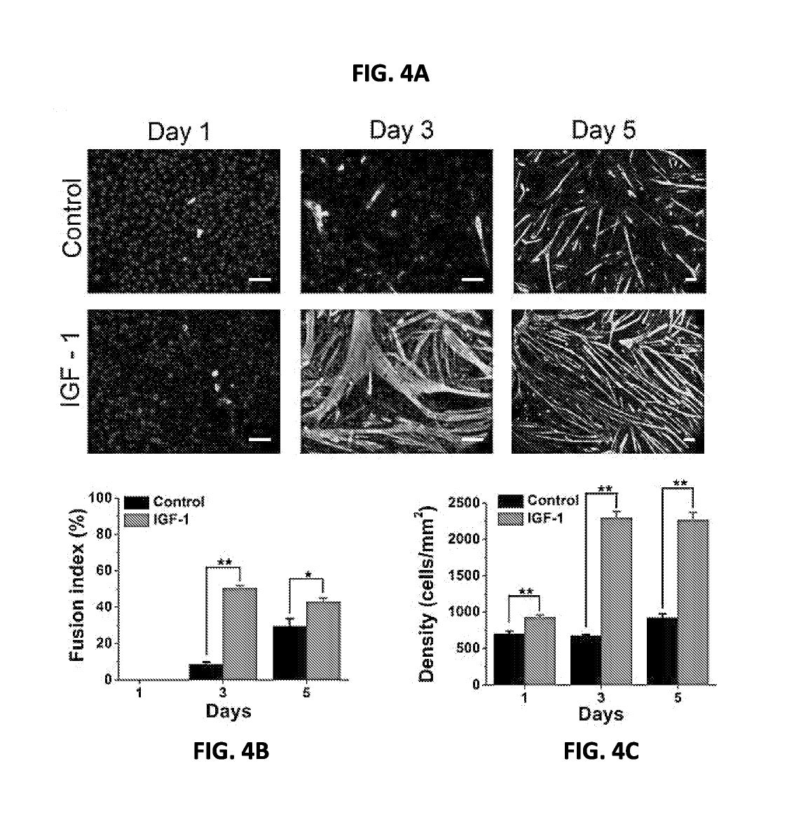

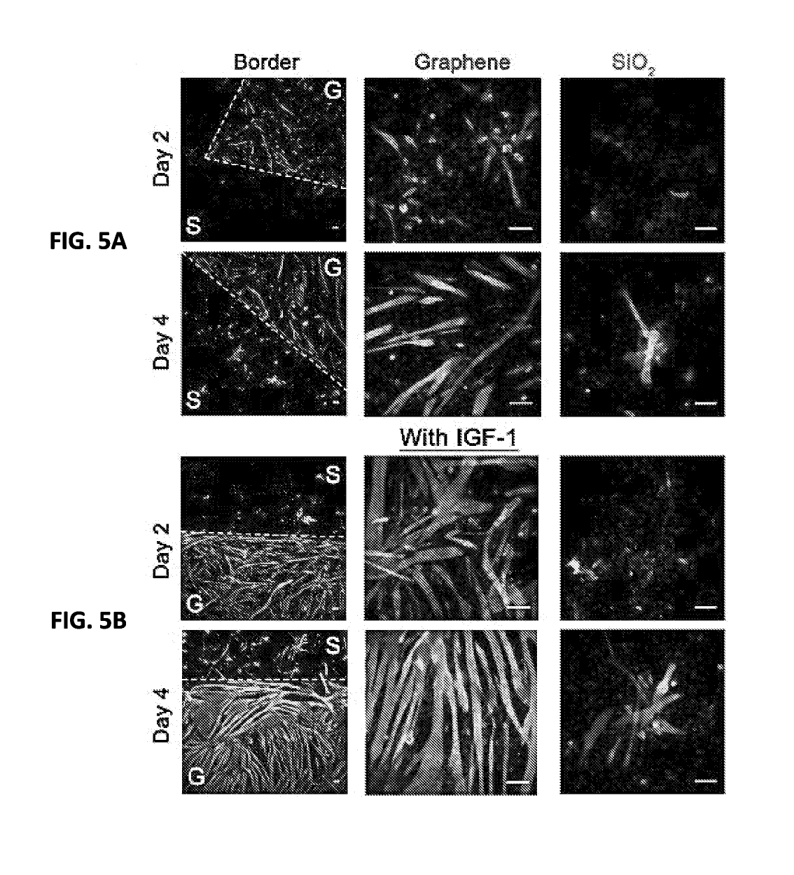

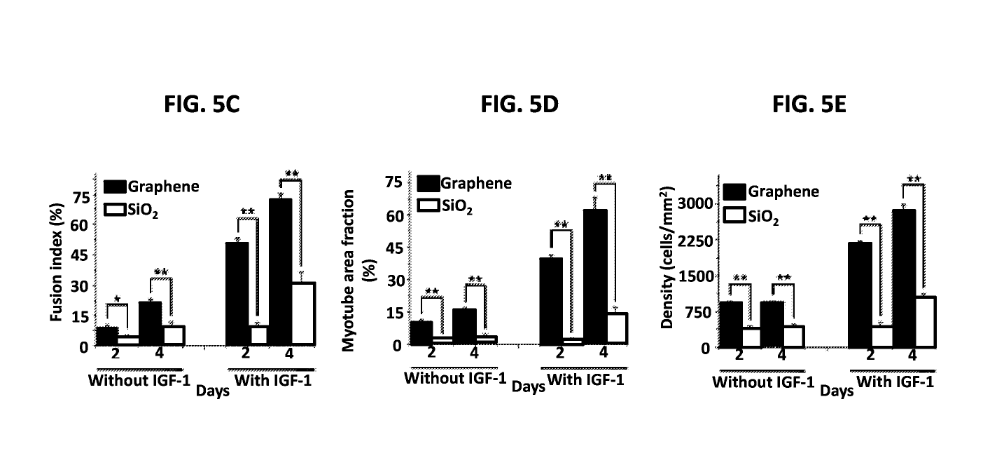

Culture of C2C12 myoblasts on graphene films in differentiation medium resulted in formation of myotubes with five days (FIG. 4A). Addition of IGF-1 to the differentiation medium significantly increased myotube formation, fusion index, and cell density (FIGS. 4A-4C). Culture of C2C12 myoblasts in differentiation medium resulted in over 50% myotubes in culture within 5 days. There was very little formation of myotubes on the SiO₂ surfaces compared to graphene (FIG. 5A) even when IGF-1 was added to the differentiation media (FIG. 5B). This is clearly seen at the border between the graphene and SiO₂ surfaces (FIGS. 5A and 5B). This is also evident in the fusion index, myotube area fraction, and cell density of cells grown on graphene as compared to SiO₂ surfaces in the presence or absence of IGF-1 (FIGS. 5C-5E). Increasing the amount of IGF-1 in the differentiation medium (from 1-500 ng/ml) increased myotube formation.

### EXAMPLE 2

### Culture of Motor Neuron Cells on Other Substrates

This example describes culture of C2C12 cells on additional substrates.

C2C12 cells were successfully cultured on graphene oxide, hexagonal boron nitride (hBN), molybdenum disulfide (MoS₂), molybdenum diselenide (MoSe₂), molybdenum ditelluride (MoTe₂), and tungsten disulfide (WS₂).

### EXAMPLE 3

### Differentiation of NSC-34 Motor Neuron Cells

This example describes differentiation of NSC-34 motor neuron cells in vitro.

NSC-34 cells (Cellutions, Cat. No. CLU 140) are a hybrid cell line produced by fusion of motor neuron enriched embryonic mouse spinal cord cells with mouse neuroblastoma, resulting in a motor neuron-like cell. NSC-34 cells were grown in high glucose DMEM with 10% FBS and 1% antibiotics. Various differentiation media were tested (formulations shown in Table 1).

TABLE 1
NSC-34 differentiation media

| Media Name | Components |
|---|---|
| DMEM/HS | High glucose DMEM + 2% HS + 1% antibiotics |
| DMEM/FBS | High glucose DMEM + 1% FBS + 1% antibiotics |
| DMEM/F12 | 1:1 DMEM/F12 + 1% FBS + 1% NEAA + 1% |
| | antibiotics |
| DMEM/F12 + RA | 1:1 DMEM/F12 + 1% FBS + 1% NEAA + 1% |
| | antibiotics + 1 μM retinoic acid |
| Neurobasal | Neurobasal media + 1% antibiotics |
| Neurobasal + N2 | Neurobasal media + N2 Supplement + 1% |
| | antibiotics |
| Neurobasal + B27 | Neurobasal media + B27 Supplement + 1% |
| | antibiotics |
| RPMI | RPMI 1640 + 10% FBS + 1% antibiotics |
| RPMI serum-free | RPMI 1640 + 1% antibiotics |

It was determined that Neurobasal medium+1% antibiotics produced optimal differentiation of NSC-34 motor neurons, therefore cells were cultured in the indicated media for 6-8 days and stained with primary antibody beta III tubulin (TUB3), followed by goat anti-mouse secondary antibody and DAPI for 2 hours at 37° C. In some examples, NSC-34 cells were cultured in Neurobasal medium with 50-100 ng/ml IGF-1. Cells were imaged with a fluorescent microscope and images were quantified using ImageJ 1.47d with the NeuronJ plugin.

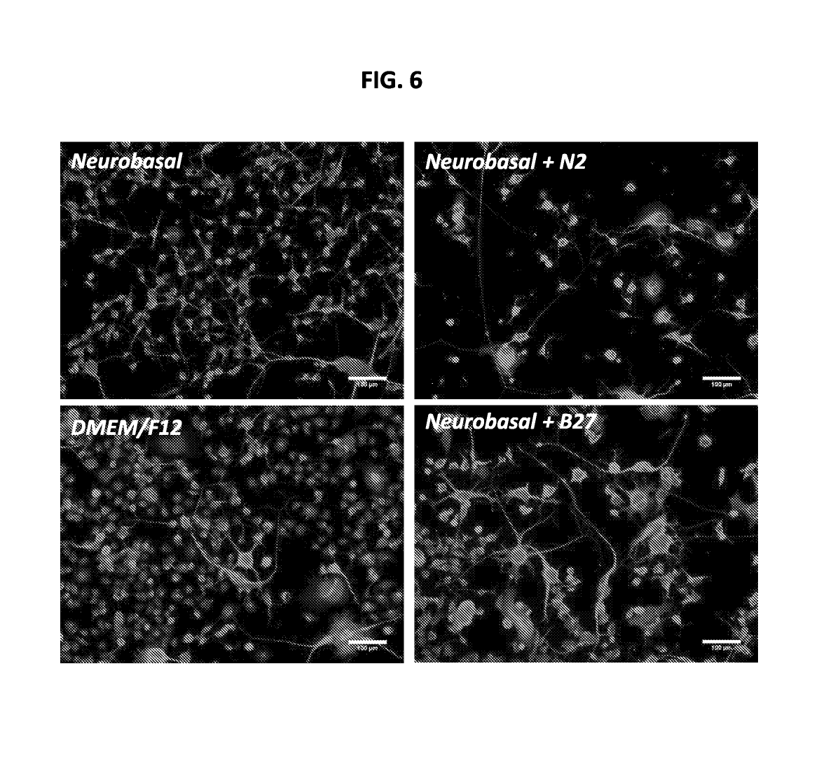

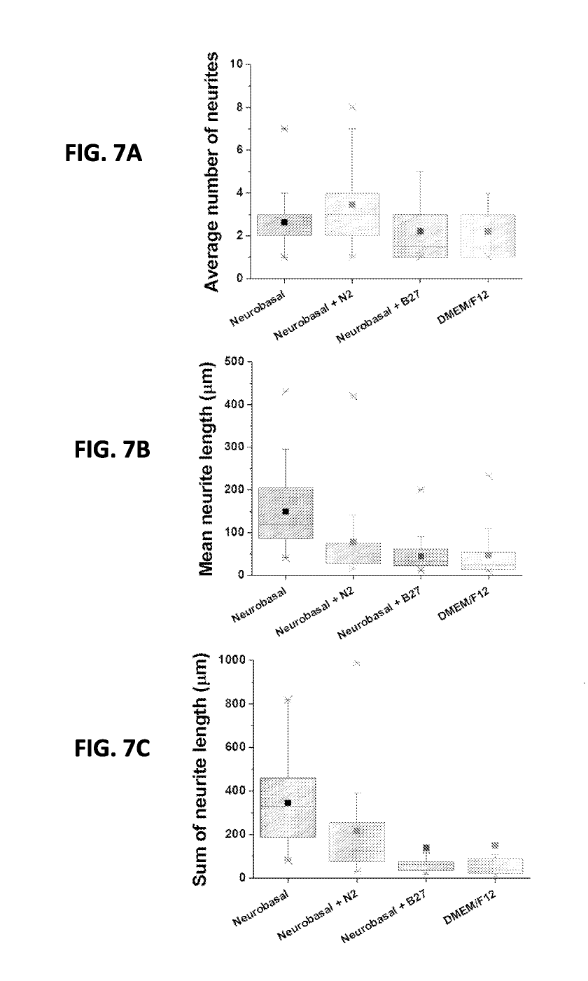

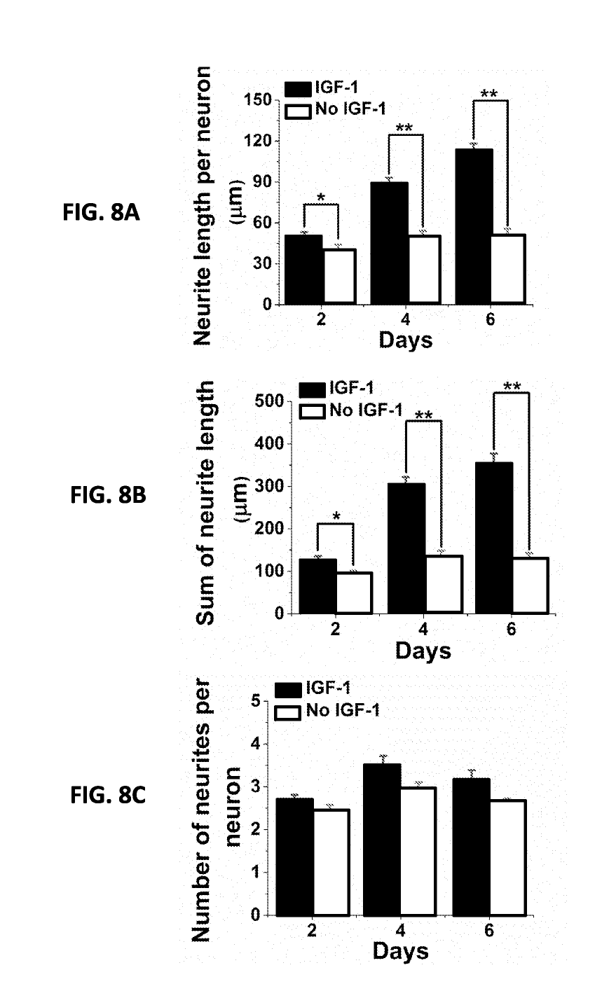

Culture of NSC-34 cells in standard culture dishes in each of DMEM/F12, Neurobasal, Neurobasal+N2, and Neurobasal+B27 differentiation media resulted in differentiation, as indicated by neurite outgrowth (FIG. 6). Cells cultured in Neurobasal+N2 media had the highest average number of neurites per neuron (FIG. 7A), while Neurobasal media resulted in the highest mean neurite length and sum of neurite length (FIGS. 7B and 7C). Addition of IGF-1 to Neurobasal differentiation media significantly increased neurite length per neuron and sum of neurite length (FIGS. 8A and 8B), but did not affect number of neurites per neuron (FIG. 8C).

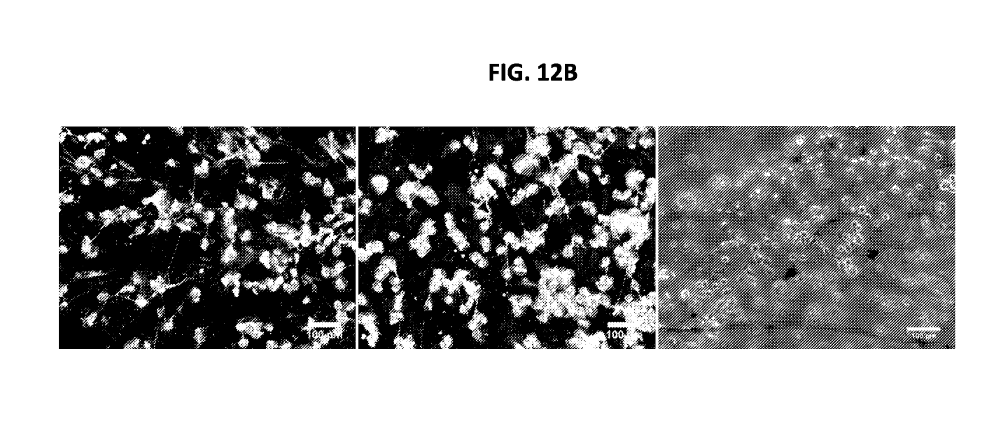

NSC-34 cells were also cultured on graphene and graphene oxide in Neurobasal medium and neurite formation was observed (FIGS. 12A and 12B). NSC-34 cells were also successfully cultured on hexagonal boron nitride (hBN), molybdenum disulfide (MoS₂), molybdenum diselenide (MoSe₂), molybdenum ditelluride (MoTe₂), and tungsten disulfide (WS₂).

### EXAMPLE 4

### Formation of Neuromuscular Junctions in Culture

This example describes co-culture of C2C12 cells and NSC-34 cells in vitro and formation of NMJs.

C2C12 cells were seeded at 40,000 cells/cm² in tissue culture dishes. After 1 day, medium was switched C2C12 differentiation medium (DMEM+2% HS) and cells were cultured for 6 days. NSC-34 cells were then seeded in the culture at 2500 cells/cm² in growth medium. The following day, the medium was switched to Neurobasal differentiation medium and cultured for 6 days. A summary of the protocol is shown in FIG. 9. The co-culture was then fixed and imaged using the indicated antibodies.

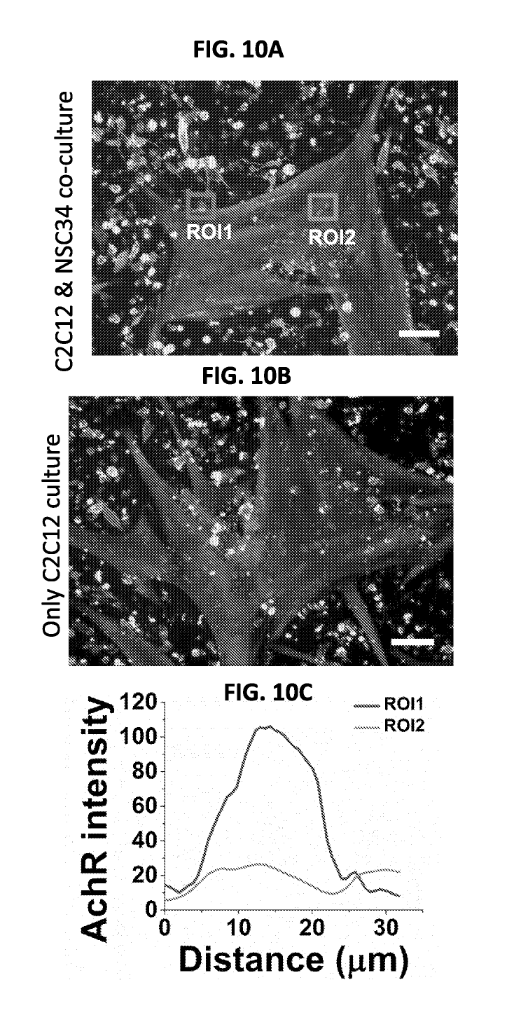

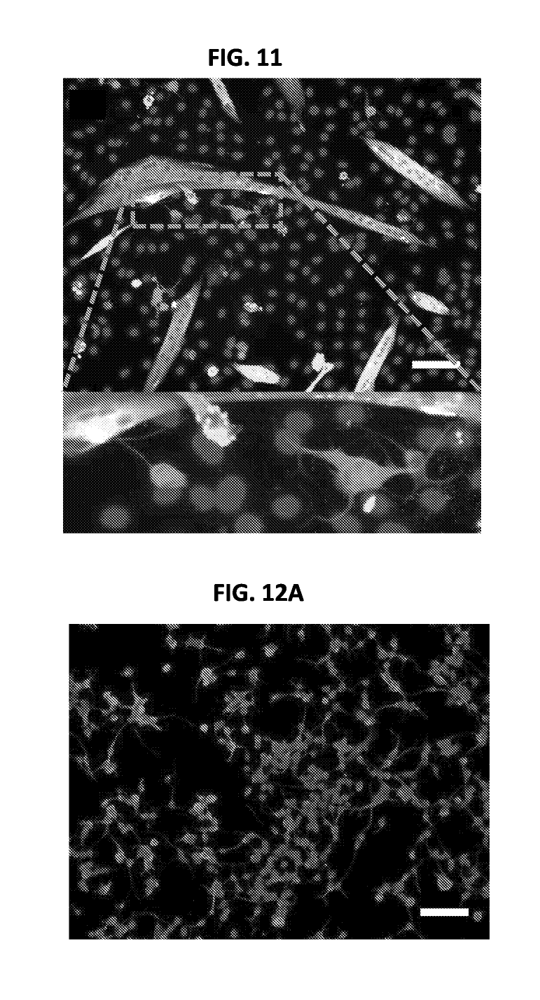

Co-culture of C2C12 and NSC-34 cells as shown in FIG. 9 resulted in formation of myotubes (C2C12 cells), neurite outgrowth (NSC-34 cells) and formation of NMJs. Formation of NMJs was indicated by presence of clusters of acetylcholine receptors (AchR) (FIGS. 10A-10C). Increased AchR clustering was observed at the site of interaction between the neurons and myotubes (FIG. 10C), possibly due to release of the AchR cluster inducing factor agrin from the neurites. NMJ formation could be clearly seen in higher power images, as evidenced by AchR clustering at sites of interaction between muscle and neurons (FIG. 11). About 2-3 NMJs were observed per 35 mm² culture dish.

### EXAMPLE 5

### Formation of Neuromuscular Junctions in on Graphene Chips

This example describes exemplary methods for formation of NMJs on graphene chips and testing of the NMJ functionality.

Graphene chips are fabricated and patterned using standard photolithography as described above. These chips are then sterilized in 70% ethanol for 2 hours and washed three times with phosphate buffered saline and dried by nitrogen. The chips are then left under the hood with UV-light overnight. C2C12 cells are seeded on these graphene chips at a density of 40,000 cells/cm² in DMEM+10% FBS+1% antibiotics and allowed to attach and grow for 24 hours. After this the media is changed to differentiation media (DMEM +2% HS+1% antibiotics) and the cells are allowed to differentiate for 6-8 days. Medium is replaced every other day. At this point NSC34 cells (optogenetic or non-optogenetic) are seeded on these graphene chips at a density of 2500-5000 cells/cm² and allowed to attach for 1 day in DMEM+10% FBS+1% antibiotics. After 24 hours, the media is replaced by neurobasal medium with 1% antibiotics and the cells are allowed to grow for 6-8 days, with formation of NMJs at the interface of the thin and thick graphene patterns.

The devices are then used to test the functionality of the so formed nascent NMJs. By using an LED array, the optogenetic neurons are stimulated to produce contraction of the C2C12 myotubes. This contraction of the myotubes is recorded by the underlying graphene FETs. Different compounds (such as toxic or non-toxic) are then introduced in the chamber with cell culture medium. The change of the chemical activity at the synapse (for example, a different electrical output) is measured from these graphene FETs.

### EXAMPLE 6

### Screening Assay for Identifying Modulators of NMJ Activity

This example describes methods that can be used to identify compounds that modulate NMJ activity using the devices and systems described herein. However, one skilled in the art will appreciate that methods that deviate from these specific methods can also be used to successfully identify compounds that modulate NMJ activity.

A graphene chip with an array of NMJs is produced, for example using the methods described in Example 5. The cells and/or the NMJs on the device are contacted with a test compound. In some examples, the cells and/or NMJs are contacted with varying concentrations of the test compound (such as 0.1 nM to 1 M). This can be carried out on separate devices, sequentially using the same device (for example, proceeding from low to high concentrations, with or without intervening wash steps), or simultaneously using the same device (such as a device with multiple microfluidic channels, as in FIG. 2E).

Following introduction of the test compound, the neuronal cells are stimulated, either by direct electrical stimulation (for example, using a stimulating electrode) or by exposure to light (if the neuronal cells express a light-activated ion channel, for example channelrhodopsin-2). The activity of the NMJ is determined by recording the electrical activity at the NMJ, muscle, and/or neuronal cells (for example through a recording electrode or from the underlying graphene FET) and/or detecting contraction of the myotubes. A test compound that alters the NMJ activity (for example, increases or decreases activity) compared to a control is identified as a compound that modulates NMJ activity and may be selected as a candidate for further in vitro or in vivo testing. One of skill in the art can select a suitable control. In some examples, the control includes cells and/or NMJs that have not been treated with the test compound. In other examples, the control includes a standard reference value or range of values (such as activity of previously tested cells and/or NMJs under standard conditions and in the absence of test compound(s)).

In additional examples, test compounds are screened to identify compounds that modulate NMJ activity in the presence of another compound (for example, a compound with a known effect on NMJ activity). The cells and/or the NMJs on the device are contacted with a selected compound (such as a known toxin, for example, an organophosphate compound). Following introduction of the selected compound, the cells and/or NMJs are contacted with a test compound (such as a candidate counter-toxin). In some examples, the cells and/or NMJs contacted with the selected compound are contacted with varying concentrations of the test compound (such as 0.1 nM to 1 M). This can be carried out on separate devices, sequentially using the same device (for example, proceeding from low to high concentrations, with or without intervening wash steps), or simultaneously using the same device (such as a device with multiple microfluidic channels, as in FIG. 2E).

The neuronal cells are stimulated, either by direct electrical stimulation (for example, using a stimulating electrode) or by exposure to light (if the neuronal cells express a light-activated ion channel, for example channelrhodopsin-2). The activity of the NMJ is determined by recording the electrical activity at the NMJ, muscle or neuronal cells (for example through a recording electrode or from the underlying graphene FET) and/or detecting contraction of the myotubes. A test compound that alters the NMJ activity in the presence of the selected compound (for example, increases or decreases activity) compared to a control is identified as a compound that modulates NMJ activity (for example, is a potential counter-toxin) and may be selected as a candidate for further in vitro or in vivo testing. One of skill in the art can select a suitable control. In some examples, the control includes cells and/or NMJs that have been treated with the selected compound, but have not been treated with the test compound. In other examples, the control includes a standard reference value or range of values (such as activity of previously tested cells and/or NMJs under standard conditions and in the presence of the selected compound).

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. A system, comprising:
a substrate comprising one or more regions of a biocompatible electrically active material disposed on the substrate wherein the regions of the electrically active material comprise at least one first area and at least one second area, wherein the second area is perpendicular or radial to the first area and is in physical contact with the first area;
at least one electrode connected to each of the one or more regions of electrically active material;
a plurality of muscle cells and a plurality of neuronal cells, wherein the muscle cells and neuronal cells are present on the one or more regions of the electrically active material; and
one or more neuromuscular junctions between the neuronal cells and muscle cells.

2. The system of claim 1, wherein the biocompatible electrically active material comprises graphene, graphene oxide, hexagonal boron nitride, molybdenum disulfide, molybdenum diselenide, molybdenum ditelluride, or tungsten disulfide and/or wherein the substrate comprises silicon/silicon dioxide.

3. (canceled)

4. The system of claim 1, wherein at least one of the regions of the electrically active material comprises a rectangular first area and two or more second areas that are oriented perpendicularly or radially to the first area and are in direct contact with the first area.

5. The system of claim 1, wherein the at least one region of electrically active material comprises two or more second areas, each of which are oriented perpendicularly or at an angle of greater than 5° with respect to the first area and are each in contact with the first area.

6. The system of claim 1, wherein the at least one region of electrically active material comprises a first area and 2-100 second areas that are each oriented perpendicularly or radially to the first area and are in physical contact with the first area

7. The system of claim 1, wherein the substrate comprises 2-3600 regions of electrically active material.

8. The system of claim 1, further comprising a container holding the substrate and/or circuitry for detecting and/or recording changes in electrical activity of the electrically active material.

9. (canceled)

10. The system of claim 1, wherein the plurality of muscle cells are present on the first area of the region of the electrically active material.

11-12. (canceled)

13. The system of claim 1, wherein the plurality of neuronal cells are present on the second area of the region of the electrically active material.

14-15. (canceled)

16. The system of claim 1, wherein the neuronal cells express an optically activated ion channel.

17. (canceled)

18. The system of claim 1, wherein the system comprises two or more neuromuscular junctions between the muscle cells and neuronal cells.

19. (canceled)

20. The system of claim 8, wherein the circuitry for detecting and/or recording changes in the electrically active material detects and/or records changes in the electrical activity of the neuronal cells or the muscle cells.

21. A method, comprising:
contacting the plurality of muscle cells and/or the plurality of neuronal cells of the system of claim 1 with one or more test compounds;
stimulating the plurality of neuronal cells; and
determining a response of one or more of the muscle cells, neuronal cells, or neuromuscular junctions.

22. The method of claim 21, wherein stimulating the plurality of neuronal cells comprises electrically stimulating the neuronal cells or wherein the plurality of neuronal cells express an optically activated ion channel and stimulating the plurality of neuronal cells comprises exposing the cells to a light stimulus.

23-24. (canceled)

25. The method of claim 21, wherein the response of one or more of the muscle cells, neuronal cells, or NMJs comprises electrical activity and/or muscle cell contractility.

26. The method of claim 21, further comprising contacting the plurality of muscle cells and/or the plurality of neuronal cells with a toxin prior to contacting with the one or more test compounds.

27-28. (canceled)

29. The method of claim 21, further comprising comparing the response of one or more of the muscle cells, neuronal cells, or neuromuscular junctions to a control.

30. The method of claim 29, wherein the control comprises a response of one or more of the muscle cells, neuronal cells, or neuromuscular junctions that have not been contacted with the test compound or a response of one or more of the muscle cells, neuronal cells, or neuromuscular junctions contacted with the toxin, but not the test compound, or wherein the control comprises a reference value.

31. (canceled)

32. A device, comprising:
a substrate comprising one or more regions of a biocompatible electrically active material disposed on the substrate wherein the regions of the electrically active material comprise at least one first area and at least one second area, wherein the second area is perpendicular or radial to the first area and is in physical contact with the first area;
at least one electrode connected to each of the one or more regions of electrically active material; and
a plurality of muscle cells and a plurality of neuronal cells on the one or more regions of the electrically active material.

33-34. (canceled)

35. The device of claim 32, wherein at least one of the regions of the electrically active material comprises a first area and two or more second areas that are each oriented perpendicularly or radially to the first area and are in physical contact with the first area.

36. The device of claim 35, wherein the at least one region of electrically active material comprises a first area and 2-100 second areas that are each oriented perpendicularly or radially to the first area and are in physical contact with the first area

37. (canceled)

38. The device of claim 32, further comprising a container holding the substrate and/or circuitry for detecting and/or recording changes in electrical activity of the electrically active material.

39-41. (canceled)
